(19) United States
(12) Patent Application Publication
(10) Pub. No.: US 2026/0053377 A1
(43) Pub. Date: Feb. 26, 2026

(54) SYSTEM FOR DETECTING BIOLOGICAL INFORMATION OF DRIVER OF MOVING OBJECT

(71) Applicant: TOYODA GOSEI CO., LTD., Kiyosu-shi (JP)

(72) Inventor: Rie ONO, Kiyosu-shi (JP)

(21) Appl. No.: 19/266,795

(22) Filed: Jul. 11, 2025

(30) Foreign Application Priority Data
Aug. 20, 2024 (JP) 2024-138512

(51) Int. Cl. A61B 5/021; A61B 5/00; A61B 5/308; B60Q 9/00

(52) U.S. Cl. CPC A61B 5/02125; A61B 5/308; A61B 5/6893; A61B 5/7282; B60Q 9/00; A61B 2560/0223

(57) ABSTRACT

A system for detecting biological information of a driver of a moving object includes: an electrocardiogram sensor provided on a steering wheel of the moving object, the electrocardiogram sensor being configured to detect an electrocardiogram signal of the driver; a pulse wave sensor provided on a seat belt of a seat of the moving object, the pulse wave sensor being configured to detect a pulse wave signal of the driver; a PTT estimation unit configured to estimate a pulse transmission time of the driver using the electrocardiogram signal and the pulse wave signal; and a blood pressure estimation unit configured to estimate at least one of a blood pressure of the driver and a variation in the blood pressure using the estimated pulse transmission time.

### CROSS REFERENCE TO RELATED APPLICATIONS

The present application claims priority to Japanese Patent Application No. 2024-138512 filed on Aug. 20, 2024, the disclosures of which are incorporated herein by reference in its entirety.

### BACKGROUND

### Field

The present disclosure relates to a system for detecting biological information of a driver of a moving object.

### Related Art

Various devices are proposed that detect biological information of a driver, using sensors disposed in a cabin. Japanese Patent Laid-Open No. 2008-279185, for example, discloses a technique in which an electrocardiogram signal and a pulse wave signal of a driver are acquired by electrodes and an optical sensor, which are disposed on a steering wheel. To be more specific, the electrodes detect the electrical signals produced by the beating heart through both hands grasping the steering wheel. In this way, the electrocardiogram signal is acquired. The optical sensor detects the intensity of reflected light that has been emitted toward and reflected from the skin of a hand gripping the steering wheel. As the blood flow in the arteries changes, the light absorption rate within the skin varies accordingly, resulting in a change in the intensity of the reflected light. By utilizing this variation in reflected light intensity, a pulse wave signal can be acquired. The blood pressure of the driver is estimated by using the electrocardiogram signal and the pulse wave signal acquired in this manner.

In the technique of Japanese Patent Laid-Open No. 2008-279185, both the electrodes and the optical sensor are disposed on the steering wheel and hence, it is necessary that the driver simultaneously contact both the electrodes and the optical sensor. However, in the case of a driver having relatively small hands, there is a possibility that the driver cannot simultaneously contact both the electrodes and the optical sensor, so that blood pressure cannot be accurately estimated.

### SUMMARY

The present disclosure can be realized by the following aspects.

According to one aspect of the present disclosure, a system for detecting biological information of a driver of a moving object is provided. This system includes: an electrocardiogram sensor provided on a steering wheel of the moving object, the electrocardiogram sensor being configured to detect an electrocardiogram signal of the driver; a pulse wave sensor provided on a seat belt of a seat of the moving object, the pulse wave sensor being configured to detect a pulse wave signal of the driver; a PTT estimation unit configured to estimate a pulse transmission time of the driver using the electrocardiogram signal and the pulse wave signal; and a blood pressure estimation unit configured to estimate at least one of a blood pressure of the driver and a variation in the blood pressure using the estimated pulse transmission time.

The present disclosure may be realized in various modes. For example, the present disclosure may be realized in a mode, such as a vehicle in which the system is mounted, a method for estimating at least one of a blood pressure and a variation in the blood pressure, or a program that executes such a method.

### BRIEF DESCRIPTION OF THE DRAWINGS

FIG. 1 is a diagram showing the schematic configuration of a system for detecting biological information of a driver of a vehicle according to one mode of the present disclosure;

FIG. 2 is a diagram showing the schematic configuration of the system for detecting biological information of the driver of the vehicle according to one mode of the present disclosure;

FIG. 3 is a block diagram showing the configuration of a control device;

FIG. 4 is a flowchart showing the procedure of blood pressure estimation processing;

FIG. 5 is a block diagram showing the configuration of a control device of a system of a second embodiment;

FIG. 6 is a block diagram showing the configuration of a control device of a system of a third embodiment;

FIG. 7 is a flowchart showing the procedure of blood pressure estimation processing performed by the system of the third embodiment;

FIG. 8 is a block diagram showing the configuration of a control device of a system of a fourth embodiment;

FIG. 9 is a flowchart showing the procedure of blood pressure estimation processing performed by the system of the fourth embodiment; and

FIG. 10 is a block diagram showing the configuration of a control device of a system of a fifth embodiment.

### DETAILED DESCRIPTION

### A. First Embodiment

### <Overall Configuration of System 1>

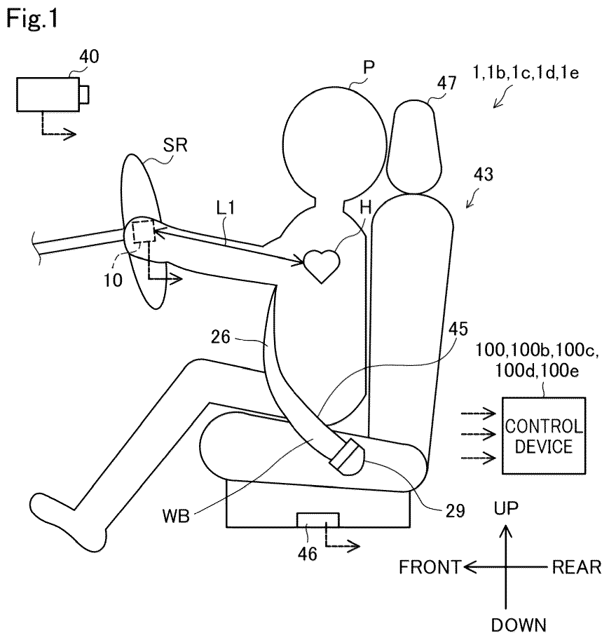

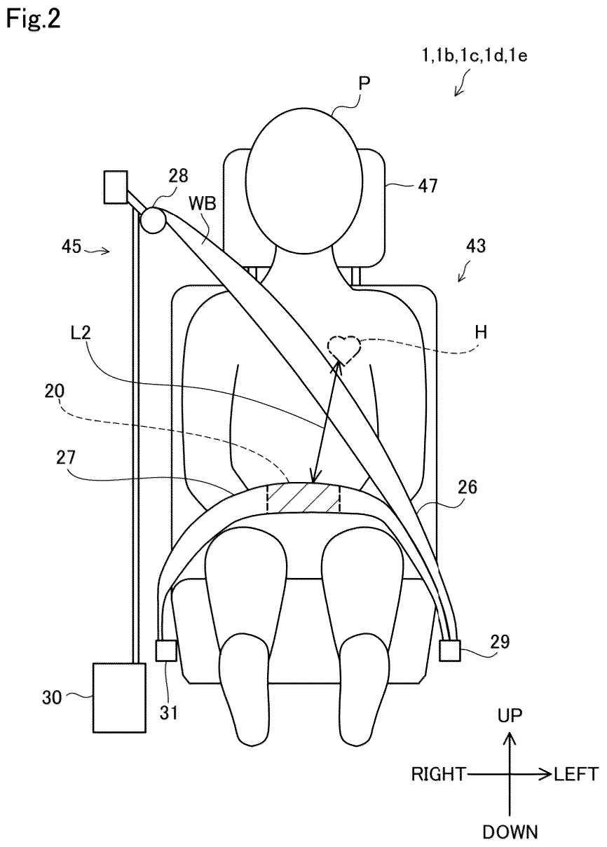

FIG. 1 and FIG. 2 are diagrams showing the schematic configurations of a system 1 for detecting biological information of a driver P of a vehicle (hereinafter also referred to as “system 1”) according to one mode of the present disclosure. FIG. 1 shows the system 1 as viewed from the left side of the vehicle, and FIG. 2 shows the system 1 as viewed from the front side of the vehicle. In FIG. 2, a steering wheel SR, an imaging device 40, and a control device 100 shown in FIG. 1 are omitted. The system 1 is used to detect information relating to blood pressure, of the biological information of the driver P of the vehicle. As shown in FIG. 1 and FIG. 2, the system 1 includes an electrocardiogram sensor 10, a pulse wave sensor 20, and the control device 100.

In the present disclosure, the direction along the advancing direction of the vehicle is referred to as “front-rear direction”. The vertical direction as viewed from the driver riding in the vehicle is referred to as “up-down direction”. The “up-down direction” may also be referred to as “height direction”. The direction along the left-right direction (width direction) of the vehicle is referred to as “left-right direction”. The “front-rear direction”, the “up-down direction”, and the “left-right direction” are directions intersecting with each other.

### <Configuration of Electrocardiogram Sensor 10>

The electrocardiogram sensor 10 shown in FIG. 1 detects the electrocardiogram signal of the driver P. The electrocardiogram sensor 10 in the present embodiment is formed of a pair of electrodes provided on the steering wheel SR. The electrocardiogram sensor 10 is provided on the rim part of the steering wheel SR, the rim part being gripped by the driver P. An electrocardiogram signal is detected when the driver P grips the pair of electrodes. The electrocardiogram signal is transmitted to the control device 100 described later.

### <Configuration of Pulse Wave Sensor 20>

The pulse wave sensor 20 shown in FIG. 2 detects the pulse wave signal of the driver P. The pulse wave sensor 20 is provided on a seat belt 45. To be more specific, the pulse wave sensor 20 is provided on the surface of a webbing WB of the seat belt 45. The webbing WB is a belt-shaped member made of polyamide or the like. The seat belt 45 in the present embodiment is a so-called three-point seat belt. The seat belt 45 includes a shoulder belt part 26 and a lap belt part 27. The shoulder belt part 26 extends from an anchor 28 to a buckle 29 in a worn state in which the seat belt 45 is worn by the driver P. The anchor 28 is a member for guiding, toward the driver P, the seat belt 45 unwound from a retractor 30. The retractor 30 is a device for winding the seat belt 45. The buckle 29 is a member provided on the lateral side of a seat 43 to fix the tongue, not shown in the drawing, of the seat belt 45. The shoulder belt part 26 covers an area from the shoulder of the driver P on one side to the abdominal part of the driver P on the other side in a worn state. The lap belt part 27 extends from the buckle 29 to an underplate 31 in the worn state. The underplate 31 is a member provided on the right side of the seat 43 to fix the seat belt 45. In the worn state, the lap belt part 27 covers the abdominal part of the driver P by crossing in the left-right direction.

In the present embodiment, the pulse wave sensor 20 is provided on the lap belt part 27, of the shoulder belt part 26 and the lap belt part 27. In the worn state, the pulse wave sensor 20 is provided on the lap belt part 27 in such a way as to be located in the vicinity of the center of the abdominal part of the driver P in the left-right direction.

The pulse wave sensor 20 in the present embodiment is a pressure sensor. The pulse wave sensor 20 captures minute motions of the body due to changes in pulse of the driver P as changes in pressure being applied to the pulse wave sensor 20. The sensor element of the pulse wave sensor 20 contains carbon nanotubes, for example. The sensor element may be manufactured by applying carbon nanotubes to the surface of the seat belt 45 by printing, or may be manufactured by sewing, onto the seat belt 45, a base material, such as a non-woven fabric, impregnated with a carbon nanotube solution. Carbon nanotubes have higher sensitivity than a conventional sensor element made of a metal material, thus allowing detection of finer pressure changes. The detected pressure change is transmitted, as a pulse wave signal of the driver P, to the control device 100 described later.

### <Configuration of Control Device 100>

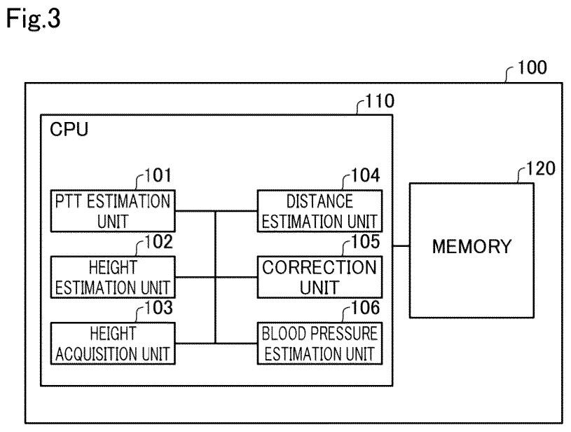

FIG. 3 is a block diagram showing the configuration of the control device 100. The control device 100 is a computer that includes a CPU 110 and a memory 120. The CPU 110 reads and executes a control program stored in advance in the memory 120 to cause a PTT estimation unit 101, a height estimation unit 102, a height acquisition unit 103, a distance estimation unit 104, a correction unit 105, and a blood pressure estimation unit 106 to execute functions.

The PTT estimation unit 101 estimates a pulse transmission time (PTT) of the driver P. The PTT is estimated using the electrocardiogram signal, which is detected by the electrocardiogram sensor 10, and using the pulse wave signal, which is detected by the pulse wave sensor 20. To be more specific, the PTT is estimated by calculating the time difference between the R wave peak of an electrocardiogram signal and the peak of a pulse wave signal.

The height estimation unit 102 estimates the height of the driver P. To be more specific, the height estimation unit 102 estimates the height of the driver P using imaging data and positional information of the seat 43, the imaging data being outputted from the imaging device 40 shown in FIG. 1, the positional information being outputted from a position sensor 46 provided on the seat 43. The imaging device 40 is provided in front of the seat 43 to image the driver P seated on the seat 43, and outputs imaging data to the height estimation unit 102. The position sensor 46 is provided on a seat rail disposed below the seat 43, and outputs positional information of the seat 43 in the front-rear direction to the height estimation unit 102. The height estimation unit 102 acquires the reference height. The reference height is a value estimated as the height of the driver P for the position of the upper end of a headrest 47 of the seat 43 matching the position of the upper end of the head of the driver P. The reference height is stored in advance in the memory 120 in association with seat positional information. Next, the height estimation unit 102 estimates the height of the driver P using the positional relationship between the upper end of the head of the driver P and the upper end of the headrest 47 in the image, using the seat positional information, and using the reference height. The relationship between a unit distance and the actual length in the image can be obtained from the positional information of the seat 43. The height estimation unit 102 converts, using this relationship, a difference between the upper end of the head and the upper end of the headrest 47 in the image into an actual length. The height estimation unit 102 estimates the height of the driver P by adding this length to the reference height, or by subtracting this length from the reference height.

The height acquisition unit 103 shown in FIG. 3 acquires height information of the driver P. The height acquisition unit 103 in the present embodiment acquires, as the height information, a height estimated by the height estimation unit 102.

The distance estimation unit 104 estimates a first distance L1 and a second distance L2 using the height information. As shown in FIG. 1, the first distance L1 is the distance from the electrocardiogram sensor 10 to a heart H with the driver P gripping the steering wheel SR. More specifically, the first distance L1 is the distance that includes a path in the body of the driver P from the electrocardiogram sensor 10 to the heart H. As shown in FIG. 2, the second distance L2 is the distance from the pulse wave sensor 20 to the heart H with the driver P wearing the seat belt 45. More specifically, the second distance L2 is the distance that includes a path in the body of the driver P from the pulse wave sensor 20 to the heart H. The first distance L1 and the second distance L2 are stored in advance in the memory 120 in association with the height information.

The correction unit 105 shown in FIG. 3 corrects, using the first distance L1 and the second distance L2, the PTT estimated by the PTT estimation unit 101. The electrocardiogram sensor 10 detects the electrocardiogram signal at the position of the hand of the driver P, and the pulse wave sensor 20 detects the pulse wave signal at the abdominal part of the driver P and hence, the electrocardiogram signal and the pulse wave signal are acquired at different positions. Therefore, the PTT estimated by the PTT estimation unit 101 may include errors. The correction unit 105 corrects the PTT using the first distance L1 and the second distance L2 to reduce errors caused by the assumption that the electrocardiogram signal and the pulse wave signal are detected at a location near the heart H. To be more specific, the correction unit 105 calculates a first transmission time using the first distance L1 and the transmission speed of the electrocardiogram signal, the first transmission time being the amount of time it takes for the electrocardiogram signal to be transmitted from the heart H to the electrocardiogram sensor 10. The correction unit 105 also calculates a second transmission time using the second distance L2 and the transmission speed of the pulse wave signal, the second transmission time being the amount of time it takes for the pulse wave signal to be transmitted from the heart H to the pulse wave sensor 20. The transmission speed of the electrocardiogram signal and the transmission speed of the pulse wave signal are stored in advance in the memory 120. The correction unit 105 calculates a correction time which is an absolute value of the difference between the first transmission time and the second transmission time. The correction unit 105 corrects the PTT by subtracting the correction time from the PTT. The corrected PTTs are stored in the memory 120 in chronological order.

The blood pressure estimation unit 106 estimates at least one of the blood pressure of the driver P and a variation in the blood pressure using the PTT corrected by the correction unit 105. The blood pressure estimation unit 106 estimates the blood pressure of the driver P using the correlation between the PTT stored in advance in the memory 120 and the blood pressure. The blood pressure estimation unit 106 also estimates the variation in the blood pressure using the PTTs of the driver P, the PTTs being stored in the memory 120 in chronological order. The term “variation in the blood pressure” includes an increase in blood pressure and a decrease in blood pressure. For example, when the PTTs decrease in chronological order, this indicates an increase in blood pressure. In contrast, when the PTTs increase in chronological order, this indicates a decrease in blood pressure. At least one of the blood pressure estimated by the blood pressure estimation unit 106 and the variation in the blood pressure may be stored in the memory 120 in chronological order.

### <Blood Pressure Estimation Processing>

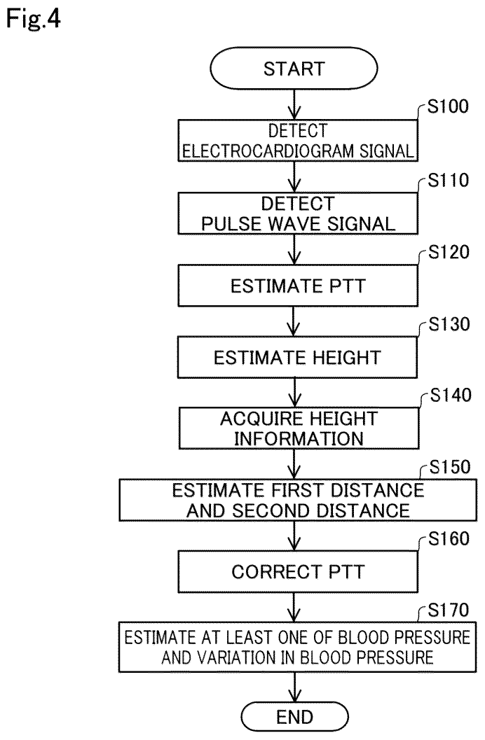

FIG. 4 is a flowchart showing the procedure of blood pressure estimation processing performed by the system 1. The blood pressure estimation processing is performed when the ignition switch of the vehicle is turned on, and the driver P wears the seat belt 45, and grips the steering wheel SR with both hands.

The electrocardiogram sensor 10 detects the electrocardiogram signal of the driver P (step S100). The pulse wave sensor 20 detects the pulse wave signal of the driver P (step S110). The PTT estimation unit 101 estimates a PTT using the electrocardiogram signal and the pulse wave signal (step S120). The height estimation unit 102 estimates the height of the driver P (step S130). The height acquisition unit 103 acquires the estimated height as height information (step S140). The distance estimation unit 104 estimates the first distance L1 and the second distance L2 using the height information (step S150). The correction unit 105 corrects the estimated PTT using the first distance L1 and the second distance L2 (step S160). The blood pressure estimation unit 106 estimates at least one of the blood pressure and the variation in the blood pressure using the corrected PTT (step S170).

The above-described blood pressure estimation processing is repeatedly performed until the ignition switch of the vehicle is turned off. Processing in step S100 and processing in step S110 may be performed in parallel.

In the system 1 of the first embodiment described above, the pulse transmission time is estimated using the electrocardiogram sensor 10, which is provided on the steering wheel SR, and using the pulse wave sensor 20, which is provided on the seat belt 45, and the blood pressure is estimated using the estimated pulse transmission time and hence, compared with a configuration in which both the electrocardiogram sensor 10 and the pulse wave sensor 20 are provided on the steering wheel SR, it is possible to estimate the blood pressure of the driver P having relatively small hands with higher accuracy.

In the system 1 of the first embodiment, the pulse wave sensor 20 is provided on the lap belt part 27, of the shoulder belt part 26 and the lap belt part 27 of the seat belt 45, and hence, compared with a configuration in which the pulse wave sensor 20 is provided on the shoulder belt part 26, it is possible to enhance accuracy of a pulse wave signal being detected. This is because the lap belt part 27 more easily comes into close contact with the body of the driver P than the shoulder belt part 26, and has relatively less opportunity of twisting.

In the system 1 of the first embodiment, the height acquisition unit 103 acquires the height information of the driver P, the distance estimation unit 104 estimates the first distance L1 and the second distance L2 using the height information, the correction unit 105 corrects the PTT using the first distance L1 and the second distance L2, and the blood pressure estimation unit 106 estimates at least one of the blood pressure and a variation in the blood pressure using the corrected PTT. Accordingly, it is possible to correct a PTT error caused by the electrocardiogram sensor 10 and the pulse wave sensor 20 located at different positions and hence, compared with a configuration in which the blood pressure estimation unit 106 estimates at least one of the blood pressure and a variation in the blood pressure using an uncorrected PTT, it is possible to estimate at least one of the blood pressure and a variation in the blood pressure more accurately.

In the system 1 of the first embodiment, the height estimation unit 102 estimates the height of the driver P using an image of the driver P and positional information of the seat 43, the image being acquired by the imaging device 40, the positional information being acquired by the position sensor 46. Accordingly, compared with a configuration that does not include the height estimation unit 102, it is possible to correct a PTT without inputting height information in advance in the system 1.

In the system 1 of the first embodiment, in the worn state, the pulse wave sensor 20 is provided on the lap belt part 27 to be located at a position in the vicinity of the center of the abdominal part of the driver P in the left-right direction and hence, compared with the case in which the pulse wave sensor 20 is located at a position offset from the center of the lap belt part 27, it is possible to reduce noise in the pulse wave signal being detected. This is because a portion of the lap belt part 27 that is located in the vicinity of the center of the abdominal part in the left-right direction more easily comes into close contact with the body of the driver P than other portions of the lap belt part 27.

In the system 1 of the first embodiment, the sensor element of the pulse wave sensor 20 contains carbon nanotubes and hence, compared with a configuration in which a sensor element does not contain carbon nanotubes, it is possible to increase sensitivity of the sensor. Consequently, the pulse wave sensor 20 can detect pulse waves with high sensitivity, even over the clothes.

In the system 1 of the first embodiment, the pulse wave sensor 20 is a pressure sensor and hence, compared with a configuration in which the pulse wave sensor 20 is an optical sensor, the pulse wave sensor 20 is less affected by ambient brightness. Therefore, the pulse wave sensor 20 can detect a pulse wave signal irrespective of the time of day and ambient brightness.

### B. Second Embodiment

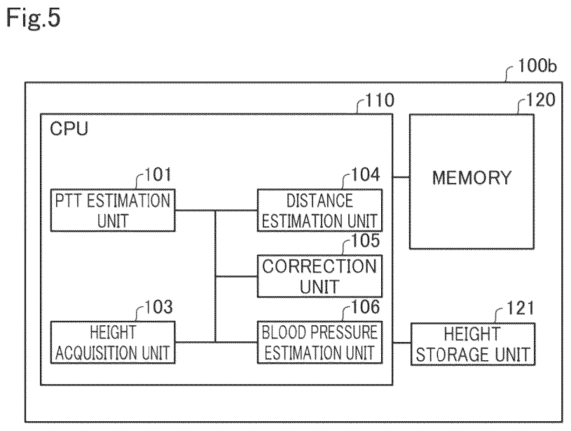

FIG. 5 is a block diagram showing the configuration of a control device 100b of a system 1b of a second embodiment. The control device 100b in the second embodiment differs from the control device 100 of the first embodiment in that the control device 100b does not include the height estimation unit 102, but further includes a height storage unit 121. Components not described below are substantially equal to the corresponding components in the system 1 of the first embodiment.

The height storage unit 121 stores the height information of the driver P. The height information may be inputted in advance through the user interface of the vehicle, or may be inputted in advance by an external device, such as a smartphone. The height storage unit 121 may store height information for each of a plurality of drivers P.

Instead of acquiring the height estimated by the height estimation unit 102, the height acquisition unit 103 in the second embodiment acquires, as the height information, height stored in the height storage unit 121. In the same manner as the first embodiment, the acquired height information is used by the distance estimation unit 104 to estimate the first distance L1 and the second distance L2.

In blood pressure estimation processing in the second embodiment, step S130 of estimating height shown in FIG. 4 is omitted. In addition, in step S140, instead of acquiring the estimated height, the height acquisition unit 103 acquires, as the height information, the height stored in the height storage unit 121.

In the system 1b of the second embodiment described above, the height storage unit 121 stores the height information of the driver P, and the height acquisition unit 103 acquires the height information stored in the height storage unit 121 and hence, the correction unit 105 can correct PTT using more accurate height information. Consequently, it is possible to further increase accuracy of the blood pressure and variation in blood pressure being estimated.

### C. Third Embodiment

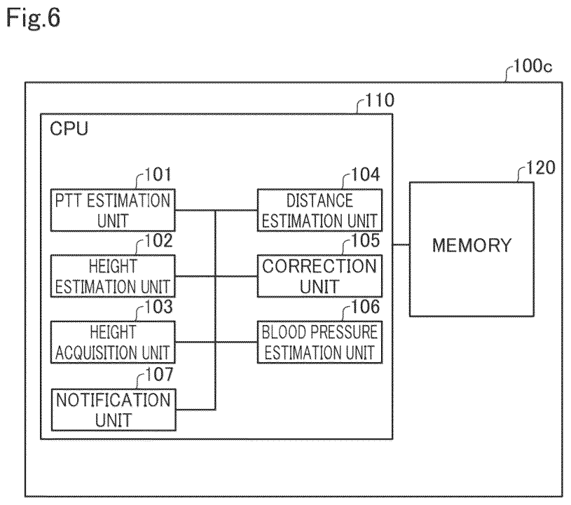

FIG. 6 is a block diagram showing the configuration of a control device 100c of a system 1c of a third embodiment. The control device 100c in the third embodiment differs from the control device 100 in the first embodiment in that the control device 100c further includes a notification unit 107. Components not described below are substantially equal to the corresponding components in the system 1 of the first embodiment.

The notification unit 107 provides a notification to the driver P when the blood pressure estimated by the blood pressure estimation unit 106 falls outside a predetermined first range. The first range is set as the blood pressure range in which the driver P is highly likely to be in a healthy physical condition. That is, when the driver P has blood pressure that falls outside the first range, the physical condition of the driver P is highly likely to be different from a healthy physical condition. The first range is set by being determined through experiments, for example. The first range is stored in advance in the memory 120. A notification is provided by sound, video, vibration, or a combination of these. Accordingly, the notification unit 107 is a speaker, a monitor, a vibration device, or the like.

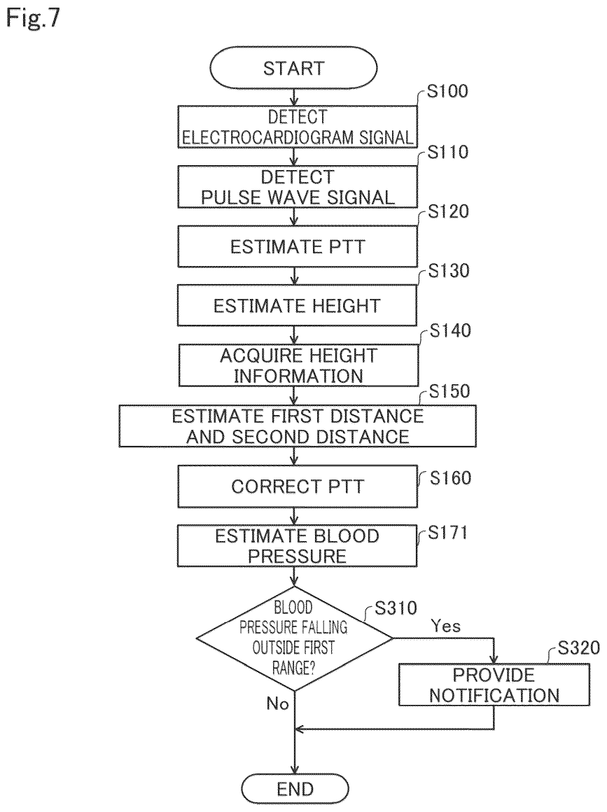

FIG. 7 is a flowchart showing the procedure of blood pressure estimation processing performed by the system 1c of the third embodiment. The blood pressure estimation processing in the third embodiment differs from the blood pressure estimation processing in the first embodiment in that processing in step S171 is performed in place of the processing in step S170, and the blood pressure estimation processing in the third embodiment further includes processing in step S310 and processing in step S320. Other steps are substantially equal to the corresponding steps of the blood pressure estimation processing in the first embodiment and hence, a description for such steps will be omitted.

As shown in FIG. 7, the blood pressure estimation unit 106 estimates the blood pressure (step S171). The blood pressure estimation unit 106 may further estimate a variation in blood pressure. That is, step S171 differs from step S170 in the first embodiment in that estimation of only variation in blood pressure does not occur.

The notification unit 107 determines whether the blood pressure falls outside the first range (step S310). The first range is predetermined prior to the processing of step S310. When the blood pressure falls outside the first range (step S310: YES), the notification unit 107 provides a notification to the driver P (step S320). When the blood pressure does not fall outside the first range (step S310: No), the blood pressure estimation processing is ended.

In the system 1c of the third embodiment described above, the notification unit 107 provides a notification to the driver P when the estimated blood pressure falls outside the predetermined first range and hence, the driver P can know the fact that the blood pressure fell outside the first range. Due to the provision of such a notification, the driver P can be made aware of the physical condition of the driver P being highly likely to be different from a healthy physical condition.

### D. Fourth Embodiment

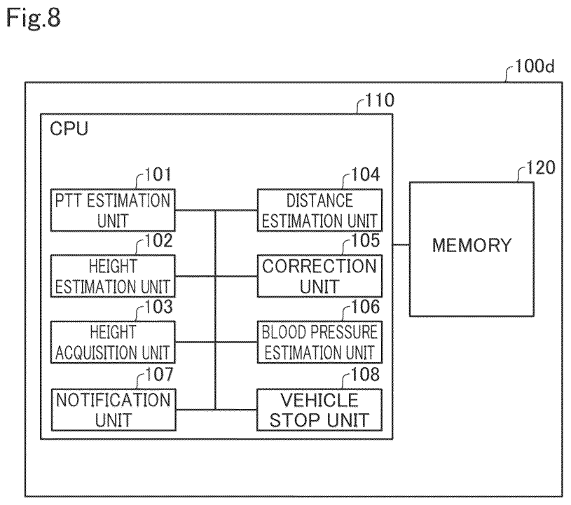

FIG. 8 is a block diagram showing the configuration of a control device 100d of a system 1d of a fourth embodiment. The control device 100d in the fourth embodiment differs from the control device 100c in the third embodiment in that the control device 100d further includes a vehicle stop unit 108. Components not described below are substantially equal to the corresponding components in the system 1c of the third embodiment.

When the CPU 110 reads and executes a control program stored in advance in the memory 120, the vehicle stop unit 108 executes a function. When the blood pressure estimated by the blood pressure estimation unit 106 falls outside a second range, which is wider than the first range, the vehicle stop unit 108 shifts the vehicle into a stopped state. The second range is set as the blood pressure range in which the driver P is highly likely to be able to continue driving. That is, when the driver P has blood pressure that falls outside the second range, the driver P is highly likely to be in a physical condition in which the driver P cannot continue driving. The second range is set by being determined through experiments, for example. The second range is stored in advance in the memory 120. When the estimated blood pressure falls outside the second range, the vehicle stop unit 108 instruct, for example, an ECU mounted in the vehicle to perform automatic operation, thus causing the vehicle to stop on the side of the road or in a parking space nearby.

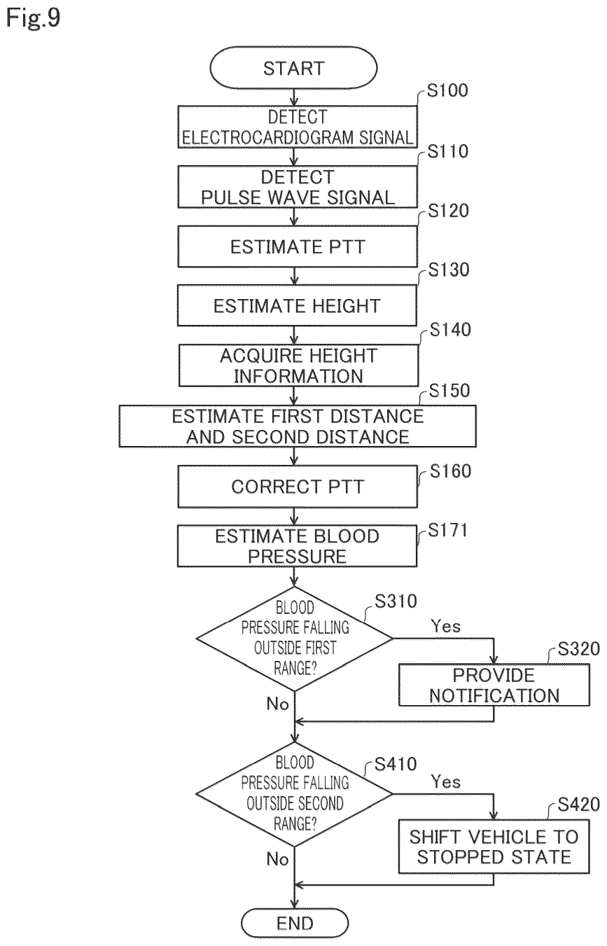

FIG. 9 is a flowchart showing the procedure of blood pressure estimation processing performed by the system 1d of the fourth embodiment. The blood pressure estimation processing in the fourth embodiment differs from the blood pressure estimation processing in the third embodiment in that the blood pressure estimation processing in the fourth embodiment further includes processing in step S410 and processing in step S420. Other steps are substantially equal to the corresponding steps of the blood pressure estimation processing in the third embodiment and hence, a description for such steps will be omitted.

As shown in FIG. 9, after the processing in step S310 or step S320, the vehicle stop unit 108 determines whether the blood pressure falls outside the second range (step S410). The second range is predetermined prior to the processing of step S310. When the blood pressure falls outside the second range (step S410: YES), the vehicle stop unit 108 shifts the vehicle into a stopped state (step S420). When the blood pressure does not fall outside the second range (step S410: No), the blood pressure estimation processing is ended.

The processing in step S410 and the processing in step S420 described above may be performed by omitting the processing in step S310 and the processing in step S320.

In the system 1d of the fourth embodiment described above, the vehicle stop unit 108 shifts the vehicle into a stopped state when the estimated blood pressure falls outside the predetermined second range and hence, it is possible to suppress the occurrence of an accident caused by a driver P being highly likely to be unable to continue driving.

### E. Fifth Embodiment

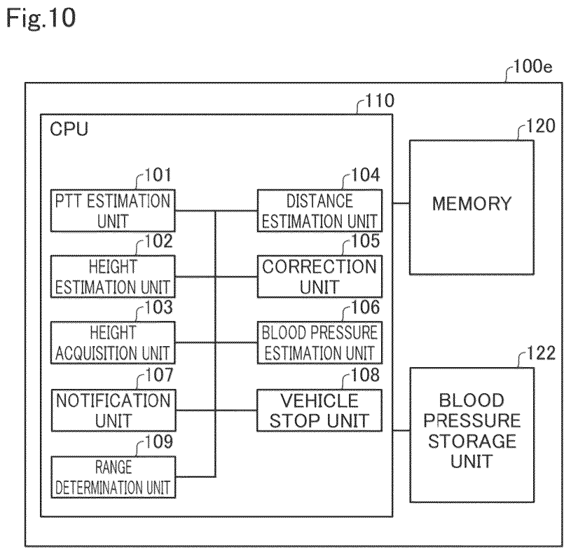

FIG. 10 is a block diagram showing the configuration of a control device 100e of a system 1e of a fifth embodiment. The control device 100e in the fifth embodiment differs from the control device 100d in the fourth embodiment in that the control device 100e includes a blood pressure storage unit 122 and a range determination unit 109.

Components not described below are substantially equal to the corresponding components in the system 1d of the fourth embodiment and hence, the description of such components will be omitted.

The blood pressure storage unit 122 stores the blood pressure estimated by the blood pressure estimation unit 106. The blood pressure is stored in such a way as to be associated with the driver P, for example. The blood pressure is stored in chronological order, for example. The blood pressure is stored in such a way as to be associated with time, for example.

When the CPU 110 reads and executes the control program stored in advance in the memory 120, the range determination unit 109 executes a function. The range determination unit 109 determines a first range and a second range using the blood pressure stored in the blood pressure storage unit 122. The range determination unit 109 determines the first range and the second range using, for example, the mean value or the median of the stored blood pressure. The range determination unit 109 calculates each of the mean value and the median of the blood pressure using, for example, blood pressure for one to two weeks, stored in chronological order. The mean value and the median of the blood pressure may be stored in advance in the blood pressure storage unit 122. Each of the first range and the second range is, for example, from a value obtained by adding a predetermined margin to the median of the blood pressure to a value obtained by subtracting a predetermined margin from the median of the blood pressure. The predetermined margin for obtaining the first range is 15 mmHG, for example. The predetermined margin for obtaining the second range is 30 mmHG, for example. Each of the first range and the second range may be determined using, for example, the mean value (μ) of the stored blood pressure and the standard deviation (σ) of the blood pressure. To be more specific, the first range is set as the range of μ±2σ. The second range is set as the range of μ±3σ. The standard deviation of the blood pressure may be calculated by the range determination unit 109 and, in addition, may be stored in advance in the blood pressure storage unit 122. The first range and the second range are not limited to the above-mentioned ranges, and any range may be set.

In the system 1e of the fifth embodiment described above, the blood pressure storage unit 122 stores the estimated blood pressure, and the range determination unit 109 determines the first range and the second range using the stored blood pressure and hence, compared with a configuration that determines the first range and the second range without using the stored blood pressure, it is possible to determine more appropriate values for the first range and the second range.

### F. Other Embodiments

(F1) The system 1b of the above-mentioned second embodiment may be combined with the system 1c of the third embodiment, the system 1d of the fourth embodiment, or the system 1e of the fifth embodiment.

(F2) In each of the above-mentioned embodiments, at least some of the components of the control device 100, 100b, 100c, 100d, 100e need not be mounted in the vehicle. That is, at least some of the components of the control device 100, 100b, 100c, 100d, 100e may be the components of a server or the like disposed outside the vehicle, and may be achieved as the system 1, 1b, 1c, 1d, 1e when this server or the like communicates with a communication device mounted in the vehicle.

(F3) In each of the above-mentioned embodiments, the pulse wave sensor 20 may be any sensor, such as a photoelectric sensor or a millimeter wave radar sensor, provided on the seat belt 45.

(F4) In each of the above-mentioned embodiments, noise removal processing may be performed on at least one of the electrocardiogram signal and the pulse wave signal. Noise is caused by, for example, vibrations resulting from the vehicle traveling. The PTT estimation unit 101 may estimate the PTT using an electrocardiogram signal and a pulse wave signal on which the noise removal processing is performed. With such a configuration, it is possible to estimate the PTT more accurately.

(F5) In the above-mentioned first embodiment, the blood pressure estimation unit 106 estimates at least one of the blood pressure and the variation in the blood pressure using the PTT corrected by the correction unit 105. However, the present disclosure is not limited to such a configuration. The blood pressure estimation unit 106 may estimate at least one of the blood pressure and a variation in the blood pressure using an uncorrected PTT.

(F6) In the above-mentioned first embodiment, the pulse wave sensor 20 is provided on the lap belt part 27 of the seat belt 45. However, the present disclosure is not limited to such a configuration. The pulse wave sensor 20 may be provided on the shoulder belt part 26 of the seat belt 45. Alternatively, the pulse wave sensors 20 may be provided on both the shoulder belt part 26 and the lap belt part 27 of the seat belt 45.

(F7) In each of the above-mentioned embodiments, the first distance L1 and the second distance L2 are estimated using the height information. However, the present disclosure is not limited to such a configuration. The first distance L1 and the second distance L2 may be stored in advance in the memory 120. The first distance L1 and the second distance L2 are stored by inputting, for example, values directly measured by the driver P, via the user interface of the vehicle. With such a configuration, it is possible to correct the PTT more accurately using a more accurate first distance L1 and a more accurate second distance L2.

(F8) In the above-mentioned third embodiment, the notification unit 107 provides a notification to the driver P when the blood pressure exceeds the first range. However, the present disclosure is not limited to such a configuration. The notification unit 107 may provide a notification to the driver P when the blood pressure exceeds a predetermined first threshold. Alternatively, the notification unit 107 may provide a notification to the driver P when the blood pressure falls below a predetermined second threshold. Each of the first threshold and the second threshold is a boundary value between the blood pressure value at which the physical condition of the driver P is highly likely to be different from a healthy physical condition and the blood pressure value at which the driver P is highly likely to be in a healthy condition. The first threshold is larger than the second threshold. When the driver P has the blood pressure exceeding the first threshold, the physical condition of the driver P is highly likely to be different from a healthy physical condition. When the driver P has the blood pressure below the second threshold, the physical condition of the driver P is highly likely to be different from a healthy physical condition. The first threshold and the second threshold are set by being determined through experiments, for example. The first threshold and the second threshold are stored in advance in the memory 120. With such a configuration, when the driver P is highly likely to have a blood pressure different from that in a healthy physical condition, it is possible to provide a notification to the driver P. The first threshold and the second threshold may be determined using blood pressure stored in the blood pressure storage unit 122. The first threshold is, for example, a value obtained by adding a predetermined margin to the median of the stored blood pressure. The first threshold is, for example, a value obtained by adding 15 mmHG to the median of the stored blood pressure. The second threshold is, for example, a value obtained by subtracting a predetermined margin from the median of the stored blood pressure. The second threshold is, for example, a value obtained by subtracting 15 mmHG from the median of the stored blood pressure. The first threshold and the second threshold may be set using the mean value (μ) of the blood pressure and the standard deviation (σ). For example, the first threshold is set as the value of μ±2σ, and the second threshold is set as the value of μ−2σ. The first threshold and the second threshold are not limited to the above-mentioned values, and any value may be set. The first threshold and the second threshold may be determined by the range determination unit 109, which executes a function thereof when the CPU 110 executes the program stored in the memory 120. The first threshold and the second threshold may be determined by a threshold determination unit not shown in the drawing, which executes a function thereof when the CPU 110 executes the control program stored in the memory 120.

(F9) In the above-mentioned fourth embodiment, the vehicle stop unit 108 shifts the vehicle into a stopped state when the blood pressure falls outside the second range. However, the present disclosure is not limited to such a configuration. The vehicle stop unit 108 may shift the vehicle into a stopped state when the blood pressure exceeds a predetermined third threshold. The vehicle stop unit 108 may shift the vehicle into a stopped state when the blood pressure falls below a predetermined fourth threshold. The third threshold is larger than the fourth threshold. Each of the third threshold and the fourth threshold is a boundary value between the blood pressure value at which the driver P is highly likely to be unable to continue driving and the blood pressure value at which the driver P is highly likely to be able to continue driving. A driver P having blood pressure exceeding the third threshold is highly likely to be unable to continue driving. A driver P having blood pressure below the fourth threshold is highly likely to be unable to continue driving. The third threshold and the fourth threshold are set through experiments, for example. The third threshold and the fourth threshold are stored in advance in the memory 120. With such a configuration, it is possible to suppress the occurrence of an accident by stopping the vehicle when the driver P is highly likely to be unable to continue driving. The third threshold and the fourth threshold may be determined using the blood pressure stored in the blood pressure storage unit 122. The third threshold is, for example, a value obtained by adding a predetermined margin to the median of the stored blood pressure. The third threshold is, for example, a value obtained by adding 30 mmHG to the median of the stored blood pressure. The fourth threshold is, for example, a value obtained by subtracting a predetermined margin from the median of the stored blood pressure. The fourth threshold is, for example, a value obtained by subtracting 30 mmHG from the median of the stored blood pressure. The third threshold and the fourth threshold may be set using the mean value (μ) of the blood pressure and the standard deviation (σ). For example, the third threshold is set as the value of μ±3σ, and the fourth threshold is set as the value of μ=3σ. The third threshold and the fourth threshold are not limited to the above-mentioned values, and any value may be set. The third threshold and the fourth threshold may be determined by the range determination unit 109. The third threshold and the fourth threshold may be determined by a threshold determination unit not shown in the drawing, which executes a function thereof when the CPU 110 executes the control program stored in the memory 120.

(F10) In each of the above-mentioned embodiments, the correction unit 105 may further correct the PTT using respiratory information of the driver. The respiratory information is information relating to the respiratory condition of the driver, and includes whether the driver is exhaling and whether the driver is inhaling. The respiratory information is acquired by, for example, a sensor provided on the seat belt 45. When the driver is exhaling, there may be cases in which the cardiac output decreases, thus causing the PTT to be longer than usual. In contrast, when the driver is inhaling, there may be cases in which the cardiac output increases, thus causing the PTT to be shorter than usual. The correction unit 105 performs a correction such that the PTT is shortened when the driver is exhaling, whereas the PTT is prolonged when the driver is inhaling. With such a configuration, a PTT error caused by respiration can be reduced and hence, the blood pressure estimation unit 106 can estimate at least one of the blood pressure and a variation in the blood pressure using a PTT that is corrected more accurately.

(F11) In each of the above-mentioned embodiments, the system 1, 1b, 1c, 1d, 1e may be provided on any moving object which is not a vehicle. Examples of such a moving object include ship, airplane, spacecraft, and so-called flying automobile. The moving object need not be a movable object, and may be for example, an object that achieves virtual movement, such as a simulator.

The present disclosure is not limited to the above-described embodiments, and may be achieved by various configurations without departing from the gist of the present disclosure. For example, to solve a part or a whole of the above-described problem or to achieve a part or a whole of the above-described effect, technical features in the embodiments may be suitably replaced or combined with each other. Further, if such a technical feature is not described in this specification as an essential technical feature, such a technical feature may be deleted when appropriate. For example, the present disclosure may be achieved by a mode described below.

(1) According to one mode of the present disclosure, a system for detecting biological information of a driver of a moving object is provided. This system includes: an electrocardiogram sensor provided on a steering wheel of the moving object, the electrocardiogram sensor being configured to detect an electrocardiogram signal of the driver; a pulse wave sensor provided on a seat belt of a seat of the moving object, the pulse wave sensor being configured to detect a pulse wave signal of the driver; a PTT estimation unit configured to estimate a pulse transmission time of the driver using the electrocardiogram signal and the pulse wave signal; and a blood pressure estimation unit configured to estimate at least one of a blood pressure of the driver and a variation in the blood pressure using the estimated pulse transmission time.

The system of this mode includes the electrocardiogram sensor provided on the steering wheel to detect an electrocardiogram signal of the driver, and the pulse wave sensor provided on the seat belt to detect a pulse wave signal of the driver, the PTT estimation unit estimates a pulse transmission time using the electrocardiogram signal and the pulse wave signal, and the blood pressure estimation unit estimates at least one of the blood pressure and a variation in the blood pressure using the estimated pulse transmission time. Accordingly, compared with a configuration in which both the electrocardiogram sensor and the pulse wave sensor are provided on the steering wheel, it is possible to estimate at least one of the blood pressure and a variation in the blood pressure with higher accuracy even when the driver has relatively small hands.

(2) In the system of the above-mentioned mode, the seat belt may include a shoulder belt part and a lap belt part, and the pulse wave sensor may be provided on the lap belt part, of the shoulder belt part and the lap belt part.

In the system of this mode, the pulse wave sensor is provided on the lap belt part, of the shoulder belt part and the lap belt part, and hence, compared with a configuration in which the pulse wave sensor is provided on the shoulder belt part, it is possible to increase accuracy of a pulse wave signal being detected. This is because the lap belt part more easily comes into close contact with the body of the driver than the shoulder belt part, and has relatively less opportunity of twisting.

(3) The system of the above-mentioned mode may further include: a height acquisition unit configured to acquire height information of the driver; a distance estimation unit configured to estimate a first distance and a second distance using the height information, the first distance being from a heart of the driver to the electrocardiogram sensor, the second distance being from the heart to the pulse wave sensor; and a correction unit configured to correct the estimated pulse transmission time using the first distance and the second distance, and the blood pressure estimation unit may estimate at least one of the blood pressure of the driver and the variation in the blood pressure using the pulse transmission time corrected.

The system of this mode includes the correction unit configured to correct the estimated pulse transmission time using the first distance and the second distance, the first distance being from the heart of the driver to the electrocardiogram sensor, the second distance being from the heart to the pulse wave sensor, and the blood pressure estimation unit estimates at least one of the blood pressure and a variation in the blood pressure using the pulse transmission time corrected. Accordingly, compared with a configuration in which the blood pressure estimation unit uses an uncorrected pulse transmission time, it is possible to estimate at least one of the blood pressure and the variation in the blood pressure with higher accuracy.

(4) The system of the above-mentioned mode may further include a height estimation unit configured to estimate a height of the driver using an image of the driver and positional information of the seat, the image being acquired by an imaging device provided on the moving object, the positional information being acquired by a position sensor provided on the seat, and the height acquisition unit may acquire, as the height information, the height estimated.

In the system of this mode, the height acquisition unit acquires, as the height information, the height estimated by the height estimation unit and hence, it is possible to omit the effort required to input height information in advance into the system.

(5) The system of the above-mentioned mode may further include a height storage unit configured to store the height information of the driver, and the height acquisition unit may acquire the height information stored in the height storage unit.

In the system of this mode, the height acquisition unit acquires the height information stored in the height storage unit and hence, it is possible to acquire more accurate height information.

(6) In the system of the above-mentioned mode, the blood pressure estimation unit may estimate the blood pressure of the driver, and the system may further include a notification unit configured to provide a notification to the driver when the blood pressure estimated falls outside a first range predetermined.

The system of this mode includes the notification unit configured to provide a notification to the driver when the blood pressure falls outside the first range and hence, the driver can be made aware of the blood pressure of the driver falling outside the first range. By setting the first range to a range from the lower limit value to the upper limit value of the blood pressure at which the driver is highly likely to be healthy, the driver to which a notification is provided can be made aware of the blood pressure of the driver falling outside the blood pressure range in which the driver is highly likely to be healthy.

(7) The system of the above-mentioned mode may further include a vehicle stop unit configured to shift the moving object into a stopped state when the blood pressure estimated falls outside a second range that is predetermined to be wider than the first range.

The system of this mode includes the vehicle stop unit configured to shift the moving object into a stopped state when the blood pressure exceeds the second range and hence, by setting the second range to a range from the upper limit value to the lower limit value of the blood pressure at which the driver is highly likely to be able to continue driving, it is possible to suppress the occurrence of an accident by shifting the vehicle into a stopped state when the blood pressure of the driver falls outside the blood pressure range in which the driver is highly likely to be able to continue driving.

(8) The system of the above-mentioned mode may further include: a blood pressure storage unit configured to store the blood pressure estimated; and a range determination unit configured to determine the first range and the second range using the blood pressure stored.

The system of this mode includes the range determination unit configured to determine the first range and the second range using the stored blood pressure and hence, compared with a configuration that uses a fixed first range and a fixed second range, it is possible to determine a more appropriate first range and second range.

What is claimed is:

1. A system for detecting biological information of a driver of a moving object, comprising:
an electrocardiogram sensor provided on a steering wheel of the moving object, the electrocardiogram sensor being configured to detect an electrocardiogram signal of the driver;
a pulse wave sensor provided on a seatbelt of a seat of the moving object, the pulse wave sensor being configured to detect a pulse wave signal of the driver;
a PTT estimation unit configured to estimate a pulse wave transit time of the driver using the electrocardiogram signal and the pulse wave signal; and
a blood pressure estimation unit configured to estimate at least one of a blood pressure of the driver and a variation in the blood pressure using the estimated pulse wave transit time.

2. The system according to claim 1, wherein
the seat belt includes a shoulder belt part and a lap belt part, and
the pulse wave sensor is provided on the lap belt part, of the shoulder belt part and the lap belt part.

3. The system according to claim 2, further comprising:
a height acquisition unit configured to acquire height information of the driver;
a distance estimation unit configured to estimate a first distance and a second distance using the height information, the first distance being from a heart of the driver to the electrocardiogram sensor, the second distance being from the heart to the pulse wave sensor; and
a correction unit configured to correct the estimated pulse transmission time using the first distance and the second distance, wherein
the blood pressure estimation unit estimates at least one of the blood pressure of the driver and the variation in the blood pressure using the pulse transmission time corrected.

4. The system according to claim 3, further comprising a height estimation unit configured to estimate a height of the driver using an image of the driver and positional information of the seat, the image being acquired by an imaging device provided on the moving object, the positional information being acquired by a position sensor provided on the seat, wherein
the height acquisition unit acquires, as the height information, the height estimated.

5. The system according to claim 3, further comprising a height storage unit configured to store the height information of the driver, wherein
the height acquisition unit acquires the height information stored in the height storage unit.

6. The system according to claim 4, wherein
the blood pressure estimation unit estimates the blood pressure of the driver, and
the system further comprises a notification unit configured to provide a notification to the driver when the blood pressure estimated falls outside a first range predetermined.

7. The system according to claim 6, further comprising
a vehicle stop unit configured to shift the moving object into a stopped state when the blood pressure estimated falls outside a second range that is predetermined to be wider than the first range.

8. The system according to claim 7, further comprising:
a blood pressure storage unit configured to store the blood pressure estimated; and
a range determination unit configured to determine the first range and the second range using the blood pressure stored.
